(19) United States
(12) Patent Application Publication
(10) Pub. No.: US 2017/0105986 A1
(43) Pub. Date: Apr. 20, 2017

(54) Topical Formulation to Enhance Genital Sensation and Function

(71) Applicant: William Andriette, Roxbury, MA (US)

(72) Inventor: William Andriette, Roxbury, MA (US)

(21) Appl. No.: 15/362,655

(22) Filed: Nov. 28, 2016

Related U.S. Application Data
(63) Continuation of application No. 14/692,555, filed on Apr. 21, 2015, now abandoned.
(60) Provisional application No. 61/981,932, filed on Apr. 21, 2014.

(51) Int. Cl. A61K 31/4525; A61K 31/7004; A61K 33/04; A61K 31/7076; A61K 31/197; A61K 31/706; A61K 9/00; A61K 47/10

(52) U.S. Cl. CPC A61K 31/4525; A61K 9/0014; A61K 31/7004; A61K 47/10; A61K 31/7076; A61K 31/197; A61K 31/706; A61K 33/04

(57) ABSTRACT

The present invention relates to a topical formulation that enhances genital sensation and function of a patient and a method for enhancing genital sensation and function of a patient, the method comprising topically applying a formulation to the genital area of the patient. The topical formulation can be utilized by either male or female users. The topical formulation incorporates active compounds which function cooperatively to stimulate nerve activity, enhancing sensitivity, and facilitating tumescence in order to increase pleasure from physical stimulation and orgasms. It has incorporated various compounds that provide energy and support energy production in a cell, that stimulate nerve activity and sensitivity, that support blood flow and/or vasodilation to a bodily region, that support penetration and transport of active compounds, and that preserve the efficaciousness, effectively dilute, serve as a carrier.

The current application is a continuation-in-part of U.S. application Ser. No. 14/692,555 filed on Apr. 21, 2015 which claims priority to the U.S. Provisional Patent application Ser. No. 61/981,932 filed on Apr. 21, 2014.

### FIELD OF THE INVENTION

The present invention relates generally to a topical formulation and its use. More specifically, the present invention is a topical formulation that is able to enhance genital sensation and function of a patient when applied to the genital region of the patient, and a method for enhancing genital sensation and function of a patient by applying an amount of the topical formulation to the patient effective to enhance genital sensation and function.

### BACKGROUND OF THE INVENTION

Currently, there are many nutritional products and medications that are aimed for sexual enhancement. Sexual enhancement formulas are provided as a means of improving sensation, genital function (e.g., tumescence), and duration of sexual activity. Existing sexual enhancement formulas are most commonly provided as an orally administered compound that is ingested by a user. One of the disadvantages of orally-administered sexual enhancers is due to their delay between ingesting the sexual enhancement formula and the onset of its effects. The delayed onset of the effects can be caused by a variety of factors, including the user's own digestive system. As well, orally ingested enhancers—even when their efficacy is desired only in the genitals—are typically distributed more systemically in the body, where they are inefficacious or can produce side-effects.

It is therefore an aim of the present invention to provide a sexual enhancement formula as a topical formulation that is applied to the penis or clitoris before sexual activity. The topical formulation incorporates active compounds which function co-operatively to stimulate nerve activity, enhancing sensitivity, and to facilitate tumescence, and to heighten orgasmic peristaltic intensity in order to increase pleasure from physical stimulation and orgasms. The present invention is intended, primarily, for individual without sexual difficulties, but the topical formulation has been shown to be beneficial for those suffering from deficiency of sexual function.

### DETAIL DESCRIPTION OF THE INVENTION

The present invention is a topical formulation that is able to enhance genital sensation and function. The topical formulation is utilized by either male or female users. The topical formulation incorporates active compounds which function cooperatively to stimulate nerve activity, enhancing sensitivity, and to facilitate tumescence and heightened orgasmic peristaltic intensity in order to increase pleasure from physical stimulation and orgasms. The present invention incorporates compounds that provide energy and/or support energy production in a cell, that stimulate nerve activity and sensitivity, that support blood flow and/or vasodilation to a bodily region, that support transdermal or transmuscosal penetration and transport of active compounds, and that preserve the efficaciousness, effectively dilute, serve as carriers, and improve the tactile properties of the aforementioned active compounds. It should be noted that active compounds may provide overlapping functionality in the formulation resulting in compounds that support more than one function of the formulation.

The present invention incorporates compounds that provide energy and/or support energy production in a cell, such as a nerve cell or muscle cell. In one embodiment of the present invention, the topical formulation selects at least one compound from a group containing D-ribose, adenosine triphosphate (ATP), a reduced form of nicotinamide adenine dinucleotide (NADH), nicotinamide riboside, mitoquinone mesylate (also named as MitoQ, mitoQ10, or mitobiquinone mesylate) and creatine.

D-ribose is the right-handed enantiomer of the five-carbon sugar ribose, which is converted for use in the pentose phosphate pathway. ATP is a phosphorylated derivative of ribose that functions as an energy-rich compound that drives a majority of intracellular activity. NADH is another phosphorylated derivate of ribose that functions as an electron acceptor. Nicotinamide riboside, a niacin-related compound found in some foods, is a precursor to the oxidized form of NAD (NAD+), helps maintain intracellular concentration of NAD, can support neuroprotection, and mitochondrial and endothelial function. Mitoquinone mesylate (mitoQ) is a mitochondrial targeted antioxidant. Creatine has been used to regenerate ATP under sudden conditions of high metabolic demand, either in the form of creatine monohydrate or the form of creatine pyruvate. The aforementioned compounds support energy production and efficiency in nerve and other cells of a user's genitalia. Specifically, nerve cells have high energy demands, particularly at their extremities, where they have high concentrations of mitochondria and high demand for mitochondrial ATP production (i.e., a mitochondrial enhancer). By providing and supporting energy production within nerve cells function and activity is improved.

The present invention incorporates nerve-stimulating compounds that, when utilized in conjunction with energy-providing and/or energy-production supporting compounds that up-regulate cells to a more metabolically active state, allow said nerve cells to upward cycle stimulation-enhancing sensations and activity (i.e. a metabolic up-regulator). In the current embodiment of the present invention, the topical formulation uses 95% piperine in the form of a standardized extract from black pepper.

Piperine is an alkaloid derived from black pepper that provides a stimulating effect when topically applied. When applied to metabolically supported nerve cells, the topical formulation is able to increase sensitivity, as for example from sensations of sexual stimulation, for instance, through activation of nerve cells' TRPV1 and TRPA1 receptors. Piperine is known broadly to be an up-regulator, for instance because of its ability, when ingested, to increase metabolism via promotion of thermogenesis. As a significant component of the metabolic rate, thermogenesis can potentially be stimulated to increase energy expenditure and fat oxidation.

The present invention incorporates blood-flow enhancing and/or vasodilating compounds that improve tumescence and blood circulation to a user's genitals. It is known that tumescence is the engorgement of erectile tissue with blood. By increasing circulation and blood flow to a user's genital region, nutrients are delivered that assist in supporting higher metabolic activity to the applied region, supporting nerve and muscle function. In one embodiment of the present invention, the topical formulation utilizes at least one of blood flow enhancing and/or vasodilating compounds from the group containing ATP, NADH, nicotinamide riboside, dimethyl isosorbide, and a black pepper extract.

ATP has been shown to signal increased blood flow to a region of a user's body when found extracellularly. NADH has been observed to work as a vasodilator when found extracellularly. Nicotinamide riboside helps activate the protein Sirtuin 1, which in turn promotes endothelial nitric oxide synthase, supportive of vasodilation. Dimethyl isosorbide, derived from corn sugar, is metabolized to produce isosorbide, which is a known vasodilating compound that is traditionally used to treating angina and congestive heart failure. Black pepper has been shown to increase blood-flow and vasodilation when topically applied and as well functions as thermogenic agent, providing a heating sensation to a user's skin, which stimulates vasodilation. Through the inclusion of the aforementioned compounds, the topical formulation is able to increase circulation to support cells and tissue with high metabolic activity while additionally providing a tumescence effect to erectile tissue in the corpora cavernosa of the penis and in the clitoris.

The present invention incorporates compounds that assist in the dermal and mucosal absorption and penetration of active compounds. In the current embodiment of the present invention, the topical formulation utilizes at least one compound from a group of dermal and mucosal absorption- and penetration-enhancing compounds containing piperine, bisabolol, dimethyl isosorbide, medium-chain triglycerides (MCT), methylsulfonyl methane (MSM), and dimethyl sulfoxide (DMSO).

Piperine has been experimentally observed to facilitate penetration and absorption of active compounds across dermal and mucosal membranes. Bisabolol is an essential oil primarily found in German chamomile that has been seen to enhance percutaneous absorption of certain large molecules. Dimethyl isosorbide functions as an epidermal penetration enhancer. Medium-chain triglycerides (MCT) have a high absorption rate with the body, which facilitates transportation of various compounds across dermal and muscosal surfaces. Through the incorporation of dermal and muscosal absorption- and penetration-enhancing compounds, the topical formulation is able to reduce the quantity of the active compounds needed to achieve an intended effect. It should be noted while the absorption- and penetration-enhancing compounds are described as being able to enhance both transdermal and transmucosal absorption and penetration, that some individual compounds may be more adept for either transdermal or transmucosal absorption and penetration. The difference is due in part to the particular properties associated of each tissue type, as well as the particular absorption and permeability properties associated with particular compounds. It is known that transmucosal penetration carries four times the absorption rate as transdermal penetration.

The present invention incorporates compounds that preserve the efficaciousness, effectively dilute, serve as carriers, and improve the tactile properties of the aforementioned compounds mentioned above. In one embodiment of the present invention, the topical formulation utilizes at least one compound from a group containing distilled water, bisabolol, dimethyl isosorbide, and a plant-based gel such as sea kelp bioferment and aloe vera. The distilled water would preserve the stability of ATP, dilute active ingredients, and facilitate topical applications of the topical formulation. The bisabolol has a secondary function that assists in the improvement of the tactile properties of the topical formulation. The dimethyl isosorbide additionally provides improved miscibility of the active ingredients.

The present invention may contain a topical formulation for enhancing genital sensation and function of a patient, the topical formulation comprising: a component of a mitochondrial enhancer comprising ATP, creatine, mitoquinone mesylate (mitoQ), nicotinamide ribose, nicotinamide adenine dinucleotide (NADH), and/or D-ribose; a component of a metabolic up-regulator comprising piperine; a component of an absorption enhancer comprising bisabolol, piperine, methylsulfonyl methane, and/or dimethyl sulfoxide; and a carrier comprising distilled water, bisabolol, and/or a plant-based gel, wherein an effective amount of the topical formulation enhances genital sensation and function in a short term after topical application to the genital area of the patient. The short term effect starts in about 4 minutes, lasts for about 20 minutes at peak intensity with a continuation glow lasting up to about 12 hours in which enhancement of sensitivity and erection is noted. The topical formulation may further comprise a component for enhancing blood flow and vasodilating being selected from the group consisting of ATP, NADH, dimethyl isosorbide, a black pepper extract, and a combination thereof.

The present invention may contain a method for enhancing genital sensation and function in a patient, the method comprising topically applying a formulation to the genital area of the patient, wherein the topical formulation comprises a component of a mitochondrial enhancer comprising ATP, creatine, mitoquinone mesylate (mitoQ), nicotinamide ribose, nicotinamide adenine dinucleotide (NADH), and/or D-ribose; a component of a metabolic up-regulator comprising piperine; a component of an absorption enhancer comprising bisabolol, piperine, methylsulfonyl methane, and/or dimethyl sulfoxide; and a carrier comprising distilled water, bisabolol, and/or a plant-based gel, wherein an effective amount of the topical formulation enhances genital sensation and function in a short term after topical application to the genital area of the patient. The short term effect starts in about 4 minutes, lasts for about 20 minutes at peak intensity with a continuation glow lasting up to about 12 hours in which enhancement of sensitivity and election is noted. The topical formulation in the method may further comprises a component of enhancing blood flow and vasodilating being selected from the group consisting of ATP, NADH, dimethyl isosorbide, a black pepper extract, and a combination thereof.

As used herein, “a” or “an” means one or more (or at least one), such as combination or active ingredients, for example, can be used in the formulations and methods of the invention. Thus, a topical formulation that comprises a component of a mitochondrial enhancer refers to a topical formulation that includes one or more of mitochondrial enhancers.

“About” is used herein to refer in the range of ±20% of the target point, for example, “about 4 minutes” means in the range of 3.2-4.8 minutes.

As used herein, “patient” means either male or female human using the topical formulation.

The Table below (Table I) shows one preferred embodiment of the present invention, which contains the essential components.

TABLE I
First Core Composition

| Compounds | Quantity | |
|---|---|---|
| D-ribose | 100 mg in 1.0 ml water | |
| Piperine | 10.0 | mg |
| Distilled water | 1.2 | ml |

In one embodiment of the present invention, the topical formulation is provided with a first core composition comprising a minimum collection of compounds that achieve the desired effects of the topical formulation. The first core composition of the topical formulation comprises the D-ribose, piperine, and distilled water. The D-ribose would enhance sensitivity and erections, while the piperine would support those factors as well as improve absorption. The distilled water would be the carrier for the formulation. The aforementioned composition can be modified and enhanced through the inclusion of some of the previously described compounds.

TABLE II
Second Core Composition

| Compounds | Quantity | |
|---|---|---|
| D-ribose | 100 mg in 1.0 ml water | |
| Piperine | 10.0 | mg |
| Adenosine Triphosphate | 15.0 | mg |
| (ATP) | | |
| Distilled water | 1.5 | ml |
| Bisabolol | 3 | drops |

In one embodiment of the present invention, the first core composition is modified and enhanced through the inclusion of various additional compounds forming the second core composition of the topical formulation (Table II). The second core composition of the topical formulation comprises D-ribose, piperine, adenosine triphosphate, bisabolol, and distilled water.

The foregoing embodiment of the topical formulation was experimentally observed to produce the desired effects. In the current embodiment of the present invention, the D-ribose in the topical formulation is expected to immediately enhance erection and sensitivity. The D-ribose is abetted by the piperine, which enhances absorption and sensitivity through its thermogenic properties. The inclusion of ATP prolongs the desired effects of the topical formulation. The effects of ATP were experimentally observed to prolong the effects of the topical formulation for about a 12-hour period following administration. The distilled water is provided in the topical formulation as a means of preserving ATP from degradation.

TABLE III
Second Core Composition and Variations

| | Formulations | | | | | | | | | |
|---|---|---|---|---|---|---|---|---|---|---|
| Compounds | Core | | 1 | | 2 | | 3 | | 4 | |
| D-ribose | 100 | mg | 100 | mg | 100 | mg | 100 | mg | 100 | mg |
| | (1.0 | ml) | (1.0 | ml) | (1.0 | ml) | (1.0 | ml) | (1.0 | ml) |
| Piperine | 10.0 | mg | 10.0 | mg | 10.0 | mg | 10.0 | mg | 10.0 | mg |
| Adenosine | 15.0 | mg | 15.0 | mg | 15.0 | mg | 15.0 | mg | 15.0 | mg |
| Triphosphate | | | | | | | | | | |
| (ATP) | | | | | | | | | | |
| Distilled water | 1.5 | ml | 1.5 | ml | 1.5 | ml | 1.5 | ml | 1.5 | ml |

| | | | | | | | | | |
|---|---|---|---|---|---|---|---|---|---|
| Bisabolol | 2 | drops | 2 | drops | — | 3 | drops | 3 | drops |

| | | | | | | |
|---|---|---|---|---|---|---|
| Nicotinamide | — | — | 30.0 | mg | — | — |
| riboside | | | | | | |

| | | | | | | |
|---|---|---|---|---|---|---|
| Creatine | — | — | — | 100 | mg | — |

| | | | | | | |
|---|---|---|---|---|---|---|
| Dimethyl sulfoxide | — | — | 2 | drops | — | — |
| (DMSO) | | | | | | |

In one embodiment of the present invention, the second core composition is modified through the inclusions various compounds (Table III). The additional compounds are provided as a means of enhancing or as an alternative means of achieving the same desired effects of the topical formulation. In one embodiment of the present invention, the nicotinamide riboside, dimethyl sulfoxide, and creatine are included to form variations of the core composition.

TABLE IV
Alternative Core Composition

| Compounds | Quantity | |
|---|---|---|
| Reduced nicotinamide adenine dinucleotide | 5.0 | mg |
| (NADH) | | |
| piperine | 10 | mg |
| Dimethyl sulfoxide (DMSO) | 2 | drops |
| Distilled water | 1.0 | ml |

In one embodiment of the present invention, the topical formulation is provided with an alternative core composition (Table IV) that primarily differs by the inclusion of the reduced form of nicotinamide adenine dinucleotide (NADH). DMSO is incorporated herein as a penetration enhancer.

TABLE V
Secondary Core Composition

| Compounds | Quantity | |
|---|---|---|
| Reduced nicotinamide adenine dinucleotide | 5.0 | mg |
| (NADH) | | |
| Piperine | 10 | mg |
| Nicotinamide riboside | 10.0 | mg |
| Distilled water | 1.5 | ml |
| Bisabolol | 2 | drops |

The foregoing alternative core composition is utilized to form a secondary core composition (Table V). The secondary core composition is distinguished from the alternative core composition by the inclusion of nicotinamide riboside. The secondary core composition relies on a synergistic relationship between the nicotinamide riboside and NADH. The synergistic relationship between nicotinamide riboside and NADH enhances erections and could possibly reduce a user's refractory period. Bisabolol is incorporated into the secondary composition as a penetration enhancer.

TABLE VI
Secondary Core Composition Variations

| | Formulations | | | | | | | |
|---|---|---|---|---|---|---|---|---|
| Compounds | Core | | 1 | | 2 | | 3 | |
| Reduced | 5.0 | mg | 5.0 | mg | 5.0 | mg | 5.0 | mg |
| nicotinamide | | | | | | | | |
| adenine | | | | | | | | |
| dinucleotide | | | | | | | | |
| (NADH) | | | | | | | | |
| Nicotinamide | 30.0 | mg | 30.0 | mg | — | | 30.0 | mg |
| riboside | | | | | | | | |
| Bisabolol | 3 | drops | 3 | drops | 3 | drops | — | |
| Distilled water | 1.0 | ml | 1.0 | ml | 1.5 | ml | 1.0 | ml |
| Creatine | — | | 150 | mg | — | | — | |
| D-ribose | — | | — | | 1.0 | ml | — | |
| Piperine | 10.0 | mg | 10.0 | mg | 10.0 | mg | 10.0 | mg |

| | | | | | |
|---|---|---|---|---|---|
| Dimethyl | — | — | — | 2 | drops |
| sulfoxide | | | | | |
| (DMSO) | | | | | |

| | | | | | |
|---|---|---|---|---|---|
| Adenosine | — | — | 15.0 | mg | — |
| triphosphate | | | | | |
| (ATP) | | | | | |

In one embodiment of the present invention, the secondary core composition is modified using creatine, D-ribose, dimethyl sulfoxide (DMSO), and ATP, to create variations of the secondary core composition (Table VI). The variation of the secondary core composition provides additional means of achieving the same or improved function of the topical formulation.

TABLE VII
Additional Compositions

| | Formulations | | | | | |
|---|---|---|---|---|---|---|
| Compounds | 1 | | 2 | | 3 | |
| D-ribose | 100 mg/1.0 ml | | 100 mg/1.0 ml | | 100 mg/1.0 ml | |
| Piperine | 10.0 | mg | 15.0 | mg | 10.0 | mg |
| Adenosine | 15.0 | mg | 15.0 | mg | 15.0 | mg |
| triphosphate | | | | | | |
| (ATP) | | | | | | |
| Distilled water | 1.5 | ml | 1.5 | ml | 1.7 | ml |
| Bisabolol | 3 | drops | 3 | drops | 3 | drops |

| | | | | |
|---|---|---|---|---|
| Reduced nicotinamide | — | — | 5.0 | mg |
| adenine dinucleotide | | | | |
| (NADH) | | | | |
| Nicotinamide riboside | — | — | 30.0 | mg |
| Creatine | — | — | 150 | mg |
| Dimethyl sulfoxide | — | — | 2 | drops |
| (DMSO) | | | | |

The present invention may provide additional formulations (Table VII) that include compounds that are known to enhance sexual function and performance. In an additional embodiment of the invention, the topical formulation may include the following compounds such as glutathione.

Glutathione, a key antioxidant (primarily produced in the liver, but capable of being produced by all cells in the body) which has been shown to contribute to nerve function where nerves are stressed by high levels of reactive oxygen species (ROS), with ROS burden increased by up-regulated metabolic activity, as one would expect during prolonged stimulation. Glutathione is used in the present invention at 20 mg per dose.

In addition, the present invention may also contains a few other components that would be helpful to enhance the efficacy of the present invention.

The various forms of coQ10, such as ubiquinone and ubiquinol also exhibit a functionality to support mitochondrial function, yet seems not functional in the present invention. However, one form of coQ10, which is bound to a lipophilic cation to facilitate absorption into the negatively-charged mitochondrial matrix, does seem to add benefits. It has been used as about 0.8 mg per dose.

Glycerin, in a small amount, such as one drop per dose, is able to add more substance to the otherwise watery serum, and it potentially has a biological role as an energy store.

The present invention may contain a liquid fraction (in a vial) and a powder fraction (in a small plastic bag). The two are mixed right before use (mainly to assure efficacy of unstable-in-solution ATP). Ingredients of the liquid fraction are: water, bisabolol, glycerin, and dimethyl sulfoxide. Ingredients of the powder fraction are: D-ribose, ATP, nicotinamide riboside, mitoquinone mesylate, and piperine.

### EXAMPLES

The following examples are used to show the short term effect of the topical formulation when applied to the genital area of the patient.

### Example 1

In an 80-year-old male with good sexual function for his age, respondent reported that use of the topical formulation (tried on two occasions with a few months apart) led to a feeling of “warmth,” strengthened erection, and heightened sensitivity for the period of arousal up to orgasm—about 15 minutes.

### Topical Formulation:

ATP—15 mg

bisabolol—2 drops

creatine pyruvate—80 mg—used one time, omitted one time

mitoquinone mesylate—0.8 mg

nicotinamide ribose—30 mg

piperine—10 mg

ribose 100 mg

dissolved in 3 ml of plant-based gel (sea kelp bioferment)

### Example 2

A 76-year-old male with diabetes and poor sexual function reported that use of topical formulation_improved sensitivity and intensity of feeling for a period of about 15 minutes, starting about 5 minutes after application.

Topical Formulation:

ATP—15 mg

bisabolol—2 drops

distilled water—3 ml

piperine—10 mg

ribose 100 mg

### Example 3

A 69-year-old male with normal sexual function reported that use of topical formulation improved sensitivity and intensity of feeling, leading to a stronger orgasm, over an approximately 15 minute period.

Topical Formulation:

bisabolol—2 drops

distilled water—3 ml

nicotinamide ribose—30 mg

piperine—10 mg

ribose 100 mg

### Example 4

A middle-aged male used preparation in various formulations over a period of 5 years starting at age 46, finding consistently improved sensitivity, strength of erection, intensity of orgasm, in the period of about 20 minutes after use. With routine use, no evidence of reduced efficacy from habituation. Some components of the forumla—ATP, nicotinamide ribose, mitoquinol mesylate—were associated with somewhat improved sexual function continuing about 10 hours after use. Mixtures of ingredients serving as mitochondrial enhancers without the piperine produced minimal effect.

Topical Formulations tested supporting the above:

ATP—15 mg or omitted

bisabolol—2 drops or 1 drop

creatine and/or creatine pyruvate—100 mg or omitted

DMSO—2 drops or omitted

glutathione—20 mg or omitted

glycerin—1 drop or omitted

NADH—5 mg or omitted

nicotinamide ribose—30 mg or omitted

mitoquinone mesylate—1 mg or omitted

piperine—10 mg or omitted

ribose 100 mg or omitted

For carrier: 3 ml of gel (sea kelp bioferment or aloe vera) or 3 ml distilled water or 1 ml distilled water and 2 ml gel.

All mitochondrial enhancers were tested alone and in various combinations with each other in the absence of piperine, leading to minimal enhancement, including with bisabolol as absorption enhancer, supporting the conclusion that it's only in combination with piperine that mitochondrial enhancers produce the short-term benefit found. All were tested alone with piperine as well. The best effects were with combinations of mitochondrial enhancers with piperine, in accord with the examples of formulations given here.

### Example 5

A 44-year-old male with normal sexual function tried three different formulations of the preparation. Found notable improvement in sensitivity, intensity of orgasm, and erection for a period of 20 minutes within minutes of application. In version of the formula with ATP and/or nicotinamide ribose found moderate enhancement of erection and sensitivity continuing for a period of about ten hours after use.

Topical Formulation:

—Formulation 1

bisabolol—1 drop

distilled water—3 ml

piperine—10 mg

ribose 100 mg

—Formulation 2

ATP—15 mg

bisabolol—1 drop

distilled water—3 ml

piperine—10 mg

ribose 100 mg

—Formulation 3

ATP—15 mg

bisabolol—1 drop

distilled water—3 ml

nicotinamide ribose—30 mg

piperine—10 mg

ribose 100 mg

### Example 6

A 32-year-old man with normal sexual function reported that use of topical formulation provided very significant improvement in sensitivity, intensity of feeling, with a stronger erection, stronger orgasm (in terms of being felt more intensely and greater peristaltic function).

Topical Formulation:

ATP—15 mg

bisabolol—1 drop

distilled water—3 ml

piperine—10 mg

ribose 100 mg

### Example 7

An 18-year-old male with normal sexual function reported that use of topical formulation improved sensitivity, intensity of orgasm, with improved peristaltic function, and reduced refractory period between closely spaced orgasms. Use of the formulation including combination of the mitochondrial enhancers but without piperine had no reported effect.

Topical Formulation:

ATP—15 mg

bisabolol—2 drops

creatine pyruvate—100 mg

mitoquinone mesylate—1 mg

nicotinamide ribose—30 mg

piperine—10 mg

ribose 100 mg

dissolved in 3 ml of plant-based gel (sea kelp bioferment)

### Example 8

An 18-year-old male with normal sexual function tried the formulation including combination of the mitochondrial enhancers but without piperine had no reported effect.

Topical Formulation:

ATP—15 mg

bisabolol—2 drops

creatine pyruvate—100 mg

nicotinamide ribose—30 mg

ribose 100 mg

dissolved in 3 ml of plant-based gel (sea kelp bioferment)

Although the invention has been described in relation to its specific embodiments, it is to be understood that many other possible modifications and variations can be made without departing from the spirit and scope of the invention as herein described.

What is claimed is:

1. A topical formulation for enhancing genital sensation and function of a patient, the formulation comprising
a component of a mitochondrial enhancer;
a component of a metabolic up-regulator comprising piperine;
a component for assisting dermal and mucosal absorption; and
a carrier,
wherein an effective amount of the topical formulation enhances genital sensation and function in a short term after topical application to the genital area of the patient, and wherein the short term effect lasts from about 4 minutes to about 12 hours.

2. The topical formulation for enhancing genital sensation and function of a patient according to claim 1, wherein the component of a mitochondrial enhancer is selected from the group consisting of D-ribose, adenosine triphosphate (ATP), creatine, a reduced form of nicotinamide adenine dinucleotide (NADH), nicotinamide riboside, mitoquinone mesylate, and a combination thereof.

3. The topical formulation for enhancing genital sensation and function of a patient according to claim 1, wherein the component of a metabolic up-regulator is piperine.

4. The topical formulation for enhancing genital sensation and function of a patient according to claim 1, wherein the formulation further comprises a component of enhancing blood flow and vasodilating being selected from the group consisting of ATP, NADH, dimethyl isosorbide, a black pepper extract, and a combination thereof.

5. The topical formulation for enhancing genital sensation and function of a patient according to claim 1, wherein the component for assisting dermal and mucosal absorption is selected from the group consisting of piperine, bisabolol, dimethyl sulfoxide, methylsulfonyl methane, and a combination thereof.

6. The topical formulation for enhancing genital sensation and function of a patient according to claim 1, wherein the carrier is selected from the group consisting of distilled water, bisabolol and a plant-based gel.

7. The topical formulation for enhancing genital sensation and function of a patient according to claim 6, wherein the plant-based gel comprises sea kelp bioferment or aloe vera.

8. The topical formulation for enhancing genital sensation and function of a patient according to claim 1, wherein the formulation comprises D-ribose, piperine, and distilled water.

9. The topical formulation for enhancing genital sensation and function of a patient according to claim 8, wherein the formulation comprises 100 mg of D-ribose in 1.0 ml of water, 10.0 mg of piperine, and 1.2 ml of distilled water.

10. The topical formulation for enhancing genital sensation and function of a patient according to claim 1, wherein the formulation comprises D-ribose, piperine, ATP; bisabolol, and distilled water.

11. The topical formulation for enhancing genital sensation and function of a patient according to claim 10, wherein the formulation comprises 100 mg of D-ribose in 1.0 ml of water, 10.0 mg of piperine, 15.0 mg of ATP, 3 drops of bisabolol and 1.5 ml of distilled water.

12. The topical formulation for enhancing genital sensation and function of a patient according to claim 10, wherein the formulation further comprises creatine.

13. The topical formulation for enhancing genital sensation and function of a patient according to claim 1, wherein the formulation comprises D-ribose, piperine, ATP, nicotinamide riboside, and distilled water.

14. The topical formulation for enhancing genital sensation and function of a patient according to claim 13, wherein the formulation further comprises dimethyl sulfoxide.

15. The topical formulation for enhancing genital sensation and function of a patient according to claim 1, wherein the formulation comprises NADH, piperine, dimethyl sulfoxide, and distilled water.

16. The topical formulation for enhancing genital sensation and function of a patient according to claim 1, wherein the formulation comprises NADH, piperine, nicotinamide riboside, bisabolol, and distilled water.

17. The topical formulation for enhancing genital sensation and function of a patient according to claim 15, wherein the formulation further comprises D-ribose, and ATP.

18. The topical formulation for enhancing genital sensation and function of a patient according to claim 16, wherein the formulation further comprises creatine.

19. The topical formulation for enhancing genital sensation and function of a patient according to claim 16, wherein the formulation further comprises dimethyl sulfoxide.

20. The topical formulation for enhancing genital sensation and function of a patient according to claim 16, wherein the formulation further comprises creatine and dimethyl sulfoxide.

21. A method for enhancing genital sensation and function of a patient, the method comprising topically applying a formulation to the genital area of the patient, wherein the topical formulation comprises
a component of a mitochondrial enhancer;
a component of a metabolic up-regulator comprising piperine;
a component of an absorption enhancer; and
a carrier,
wherein an effective amount of the topical formulation enhances genital sensation and function in a short term after topical application to the genital area of the patient, and wherein the short term effect lasts from about 4 minutes to about 12 hours.

22. The method for enhancing genital sensation and function of a patient according to claim 21, wherein the component of a mitochondrial enhancer is selected from the group consisting of D-ribose, adenosine triphosphate (ATP), creatine, a reduced from of nicotinamide adenine dinucleotide (NADH), nicotinamide riboside, mitoquinone mesylate, and a combination thereof.

23. The method for enhancing genital sensation and function of a patient according to claim 21, wherein the component of a metabolic up-regulator is piperine.

24. The method for enhancing genital sensation and function of a patient according to claim 21, wherein the formulation further comprises a component of enhancing blood flow and vasodilating being selected from the group consisting of ATP, NADH, dimethyl isosorbide, a black pepper extract, and a combination thereof.

25. The method for enhancing genital sensation and function of a patient according to claim 21, wherein the component for assisting dermal and mucosal absorption is selected from the group consisting of piperine, bisabolol, dimethyl sulfoxide, methylsulfonyl methane, and a combination thereof.

26. The method for enhancing genital sensation and function of a patient according to claim 21, wherein the carrier is selected from the group consisting of distilled water, bisabolol, and a plant-based gel.

27. The method for enhancing genital sensation and function of a patient according to claim 26, wherein the plant-based gel comprises sea kelp bioferment or aloe vera.

28. The method for enhancing genital sensation and function of a patient according to claim 21, wherein the formulation comprises D-ribose, piperine, and distilled water.

29. The method for enhancing genital sensation and function of a patient according to claim 28, wherein the formulation comprises 100 mg of D-ribose in 1.0 ml of water, 10.0 mg of piperine, and 1.2 ml of distilled water.

30. The method for enhancing genital sensation and function of a patient according to claim 21, wherein the formulation comprises D-ribose, piperine, ATP; bisabolol, and distilled water.

31. The method for enhancing genital sensation and function of a patient according to claim 30, wherein the formulation comprises 100 mg of D-ribose in 1.0 ml of water, 10.0 mg of piperine, 15.0 mg of ATP, 3 drops of bisabolol and 1.5 ml of distilled water.

32. The method for enhancing genital sensation and function of a patient according to claim 30, wherein the formulation further comprises creatine.

33. The method for enhancing genital sensation and function of a patient according to claim 21, wherein the formulation comprises D-ribose, piperine, ATP, nicotinamide riboside, and distilled water.

34. The method for enhancing genital sensation and function of a patient according to claim 33, wherein the formulation further comprises dimethyl sulfoxide.

35. The method for enhancing genital sensation and function of a patient according to claim 21, wherein the formulation comprises NADH, piperine, dimethyl sulfoxide, and distilled water.

36. The method for enhancing genital sensation and function of a patient according to claim 21, wherein the formulation comprises NADH, piperine, nicotinamide riboside, bisabolol and distilled water.

37. The method for enhancing genital sensation and function of a patient according to claim 35, wherein the formulation further comprises D-ribose, and ATP.

38. The method for enhancing genital sensation and function of a patient according to claim 36, wherein the formulation further comprises creatine.

39. The method for enhancing genital sensation and function of a patient according to claim 36, wherein the formulation further comprises dimethyl sulfoxide.

40. The method for enhancing genital sensation and function of a patient according to claim 36, wherein the formulation further comprises creatine and dimethyl sulfoxide.

41. The topical formulation for enhancing genital sensation and function of a patient according to claim 1, wherein the formulation further comprises an additional compound being selected from the group consisting of glutathione, ubiquinone, glycerin, and a combination thereof.

42. The method for enhancing genital sensation and function of a patient according to claim 21, wherein the formulation further comprises an additional compound being selected from the group consisting of glutathione, ubiquinone, glycerin, and a combination thereof.
